(19) United States
(12) Patent Application Publication
(10) Pub. No.: US 2004/0254668 A1
(43) Pub. Date: Dec. 16, 2004

(54) Macro-porous hydroxyapatite scaffold compositions and freeform fabrication method thereof

(76) Inventors: Bor Z. Jang, Fagro, ND (US); Laixia Yang, Fargo, ND (US)

Correspondence Address: Bor Z Jang, 2902, 28 AVE., S.W., FARGO, ND, 58103, US

(21) Appl. No.: 10/461,167

(22) Filed: Jun. 16, 2003

(51) Int. Cl. G06F 19/00; A61F 2/28

(52) U.S. Cl. 700/119; 623/23.5; 623/23.56

(57) ABSTRACT

A solid freeform fabrication method and composition for preparing a calcium phosphate-based macro-porous scaffold for tissue engineering applications. The method includes (A) preparing a mixture of dry solid powder particles in a powder container; (B) preparing a fluid component in a reservoir separate from the powder container; wherein the powder mixture and the fluid component, separately or in combination, comprise at least a calcium source and a phosphoric acid source; (C) operating a material deposition system comprising a liquid deposition device for dispensing the fluid component from the reservoir and a solid powder-dispensing device for dispensing the solid powder mixture from the powder container to selected locations on a target surface of an object-supporting platform, wherein the dispensed fluid and dispensed powder components react to form a calcium phosphate composition (particularly hydroxyapatite or its derivative); and (D) during the operating step (C), moving the deposition system and the object-supporting platform relative to one another in X-Y-Z directions to form the scaffold containing macro pores, greater than 50 μm in size.

### FIELD OF THE INVENTION

The present invention concerns the preparation of macro-porous scaffolds from novel hydroxyapatite compositions for tissue engineering, particularly through the use of solid freeform fabrication.

### BACKGROUND OF THE INVENTION

Tissue engineering (bone, cartilage, or other tissues regeneration by autogenous cell/tissue transplantation) is one of the most promising technologies in biomedical engineering. A primary goal of tissue engineering research is the development of effective techniques to repair, transplant, replace, or regenerate damaged or diseased tissues by manipulating cells, creating artificial implants, or synthesizing laboratory-grown substitutes. The “tissue induction” process, as a regenerative tissue engineering approach, involves implanting polymer or mineral scaffolds without cells in a patient. In this process, tissue generation occurs through ingrowth of surrounding tissue into the scaffold. The “cell transplantation” approach involves seeding scaffolds with cells, cytokines, and other growth-related molecules and then culturing and implanting these constructs to induce the growth of new tissue. Cultured cells are infused in a biodegradable or non-biodegradable scaffold, which may either be implanted directly in the patient or be placed in a bio-reactor (in-vitro) to allow the cells to proliferate before the tissue is implanted in the patient. Alternatively, the cell-seeded scaffold may be directly implanted. In this case the patient's body acts as an in-vivo bio-reactor. Once implanted, in-vivo cellular proliferation occurs and, in the case of bio-resorbable scaffolds, concomitant bioabsorption of the scaffold proceeds. In both the tissue induction and cell transplantation approaches, the scaffold must be biocompatible, such that it does not induce an adverse immune response from the patient or result in toxicity to the patient.

The purpose of using a scaffold is to support cells, which, after being seeded into the scaffold, cling to the interstices of the scaffold and replicate, produce their own extra-cellular matrices, and organize into the target tissue. The scaffold must be highly porous with an interconnected pore network for cell growth and flow transport of nutrients and metabolic waste. It must also have suitable surface chemistry for cell attachment, proliferation, and differentiation. In many potential clinical applications, mechanical integrity (stiffness and strength) of a scaffold is a critical factor that affects the success or failure of the implanted scaffold. Specifically, in vivo, the scaffold structure should protect the inside of the pore network proliferating cells and their extracellular matrix from being mechanically overloaded for a sufficiently long period of time. Further, the scaffold must have mechanical properties to match those of the tissues at the site of implantation.

There exist numerous techniques for manufacturing scaffolds for tissue generation. One technique, known as fiber bonding, involves preparing a mold in the shape of the desired scaffold and placing fibers, such as polyglycolic acid (PGA) into the mold and embedding the PGA fibers in a solution of poly (L-lactic acid) (PLLA) and methylene chloride. Other fabrication techniques, such as solvent-casting and particulate-leaching, melt molding, extrusion in combination with particulate leaching, emulsion freeze-drying, phase separation, and supercritical-fluid technology, have been commonly used.

Most recent techniques of scaffold fabrication make use of rapid prototyping (RP), layer manufacturing (LM), or solid freeform fabrication (SFF) technologies such as 3-D printing and fused deposition modeling (FDM). Layer manufacturing builds an object layer by layer or point by point. This process begins with creating a Computer Aided Design (CAD) file to represent the image or drawing of a desired object such as a scaffold. This object image file is further sliced into a large number of thin layers with the contours of each layer being defined by a plurality of line segments or data points connected to form polylines. The layer data are converted to tool path data normally in terms of computer numerical control (CNC) codes such as G-codes and M-codes. These codes are then utilized to drive a fabrication tool for building an object layer by layer. The SFF technology enables direct translation of the CAD image data into a 3-D physical object. The technology has enjoyed a broad array of applications such as verifying CAD database, evaluating design feasibility, testing part functionality, assessing aesthetics, checking ergonomics of design, aiding in tool and fixture design, creating conceptual models and sales/marketing tools, generating patterns for investment casting, reducing or eliminating engineering changes in production, and providing small production runs.

For tissue engineering applications, the SFF technique is advantageous in that it allows researchers to custom-design and fabricate scaffolds of a complex shape with a completely interconnected pore network in a net-shape fashion without post-SFF trimming. However, state-of-the-art SFF techniques and related materials suffer from the following shortcomings:

1. Most of the SFF techniques and the associated materials used do not provide scaffolds with adequate mechanical strength and integrity. For instance, FDM is normally limited to the fabrication of objects from wax or plastics (such as ABS and nylon).

2. Although a mixture of ceramic and resin components can be made into a continuous filament or strand form for use in a FDM process, the as-fabricated part has to go through a high temperature resin removal and sintering procedure, which is slow and could generate undesirable decomposition products (hence, unsuitable for an office or clinical environment). The high temperature requirements imposed upon the FDM process itself and the subsequent resin removal and sintering procedures make it impossible to use FDM for co-depositing scaffold materials, seeded living cells, cell growth factors and other bio-active agents. Further, FDM has a limited part resolution, down to approximately 200 μm.

3. The parts fabricated by a commercial 3-D printer (the MIT process licensed to Z Corp.) also typically have to go through resin binder removal and ceramic sintering. Without sintering, a scaffold composed of macro pores and loosely bound ceramic particles like conventionally made hydroxylapatite (HAP) would be of low mechanical strength. Hence, the 3-D printing process is not an ideal choice for scaffold fabrication from conventional HAP compositions in an office or clinical environment. In actual practice, the 3-D printer involves feeding a complete layer of powder at a time and a portion of the powder particles ends up being scraped or wasted. This is quite costly if expensive materials such as HAP powder are used in the process.

4. The conventional HAP, as a bone implant material, is normally available in two forms: block and paste. A sintered block may be machined into a desired shape, but normally with a great level of difficulty due to the brittleness of the ceramic material. A paste, prepared by mixing powder ingredients with a liquid lubricant, can be formed into a desired shape by using a mold. In some cases, this molded shape still requires high temperature sintering, subsequent to molding, to achieve a sufficient strength. It would be advantageous to prepare HAP in a precursor form that can be readily converted into a net shape in an automated fashion, without an operator's intervention or the dependence on a shaping mold. Specifically, It would be most advantageous if this HAP precursor is directly manufacturable by a SFF technique into a net-shape scaffold for tissue engineering.

Hydroxyapatite (HAP) materials are known to exhibit the basic properties of human bones and teeth. A considerable amount of research has been conducted on the remineralization of incipient dental lesions by deposition of hydroxyapatite, Ca₁₀(PO₄)₆ (OH)₂, on such lesions. Calcium-based implants also have been used for the replacement of skeletal tissues. In addition to HAP, a number of other calcium phosphate minerals, such as fluorapatite, octacalcium phosphate, whitlockite, brushite and monetite, are also known to be relatively bio-compatible.

Apatite is a particularly interesting class of materials for biomedical applications. The term “apatite” refers to a wide range of compounds represented by the general formula M²⁺₁₀(ZO₄³⁻)₆Y⁻₂, where M is a metal atom (particularly an alkali or alkaline earth atom), ZO₄ is an acid radical, where Z may be phosphorous, arsenic, vanadium, sulphur, silicon, or may be substituted in whole or in part by carbonate (CO₃²⁻), and Y is an anion (usually halide, hydroxy, or carbonate). When ZO₄³⁻ is partially or wholly replaced by trivalent anions (such as CO₃²⁻) and/or Y⁻ is partially or wholly replaced by divalent anions, then charge balance may be maintained in the overall structure by the presence of additional monovalent cations (such as Na⁺) and/or protonated acid radicals (such as HPO₄²⁻).

Among the apatite group, hydroxyapatite (HAP) and its various derivatives or variants, have been recognized to be a major structural component of biological tissues (e.g., bone, teeth, and some invertebrate skeletons). Hence, HAP is considered to be an excellent candidate material for a scaffold that is intended to be transplanted into a patient's body. It is desirable for the apatite-based scaffold to perform other functions of natural bone such as (a) to accommodate stem cells; (b) to allow infiltration by cells normally resident in natural bone such as osteoclasts and osteoblasts; (c) to allow remodeling of the material by the infiltrating cells followed by new bone in-growth; and (d) to act in metabolic calcium exchange in a manner similar to native bone.

The following relevant U.S. patents are representative of the state-of-the-art for the field of hydroxyapatite, carbonated hydroxyapatite, and their derivatives or variants:

1. R. O'Leary et al., “Flowable Demineralized Bone Powder Composition and Its Use in Bone Repair”, U.S. Pat. No. 5,073,373 (Dec. 17, 1991).

2. I. Ison et al., “Storage Stable Calcium Phosphate Cements”, U.S. Pat. No. 6,053,970 (Apr. 25, 2000).

3. M. Sumita, “Composition for Forming Calcium Phosphate Type Setting Material and Process for Producing Setting Material”, U.S. Pat. No. 5,281,404 (Jan. 25, 1994).

4. L. Chow, “Calcium Phosphate Hydroxyapatite Precursor and Methods for Making and Using the Same”, U.S. Pat. No. 5,695,729 (Dec. 9, 1997).

5. W. Brown et al., “Combinations of Sparingly Soluble Calcium Phosphates in Slurries and Pastes as Mineralizers and Cements”, U.S. Pat. No. Re. 33, 161 (Feb. 6, 1990).

6. W. Brown et al., “Dental Restorative Cement Pastes”, U.S. Pat. No. Re. 33,221 (May 22, 1990).

7. L. Chow et al., “Calcium Phosphate Hydroxyapatite Precursor and Methods for Making and Using the Same”, U.S. Pat. No. 5,522,893 (Jun. 4, 1996).

8. L. Chow et al., “Self-Setting Calcium Phosphate Cements and Methods for Preparing and Using Them”, U.S. Pat. No. 5,525,148 (Jun. 11, 1996).

9. L. Chow et al., “Calcium Phosphate Hydroxyapatite Precursor and Methods for Making and Using the Same”, U.S. Pat. No. 5,545,254 (Aug. 13, 1996).

10. L. Chow et al., “Calcium Phosphate Hydroxyapatite Precursor and Methods for Making and Using the Same”, U.S. Pat. No. 6,325,992 B1 (Dec. 4, 2001).

11. B. Constantz, “In Situ Calcium Phosphate Minerals Method”, U.S. Pat. No. 4,047,031 (Sep. 10, 1991).

12. B. Constantz, et al., “Intimate Mixture of Calcium and Phosphate Sources as Precursor to Hydroxyapatite”, U.S. Pat. No. 5,053,212 (Oct. 1, 1991).

13. B. Constantz, “Methods for In Situ Prepared Calcium Phosphate Minerals”, U.S. Pat. No. 5,129,905 (Jul. 14, 1992).

14. B. Constantz, et al., “Situ Prepared Calcium Phosphate Composition and Method”, U.S. Pat. No. 5,336,264 (Aug. 9, 1994).

15. B. Constantz, “Carbonated Hydroxyapatite Compositions and Uses”, U.S. Pat. No. 5,900,254 (May 4, 1999).

16. B. Constantz, “Paste Compositions Capable of Setting into Carbonated Apatite”, U.S. Pat. No. 5,952,010 (Sep. 14, 1999).

17. B. Constantz, “Carbonated Hydroxyapatite Compositions and Uses”, U.S. Pat. No. 5,962,028 (Oct. 5, 1999).

18. B. Constantz et al., “Kits for Preparing Calcium Phosphate Minerals”, U.S. Pat. No. 6,002,065 (Dec. 14, 1999).

19. B. Constantz, “Paste Compositions Capable of Setting into Carbonated Apatite”, U.S. Pat. No. 6,334,891 (Jan. 1, 2002).

20. P. Brown, “Bone Substitute Composition Comprising Hydroxyapatite and a Method of Production Therefor”, U.S. Pat. No. 6,201,039 (Mar. 13, 2001).

21. H. Yamazaki et al., “Method of Manufacturing Hydroxyapatite and Aqueous Solution of Biocompounds at the Same Time”, U.S. Pat. No. 6,149,796 (Nov. 21, 2000).

22. U. Ripamonti et al., “Biomaterial and Bone Implant for Bone Repair and Replacement”, U.S. Pat. No. 6,302,913 (Oct. 16, 2001).

23. K. Marra et al., “Biocompatible Compositions and Methods of Using Same”, U.S. Pat. No. 6,165,486 (Dec. 26, 2000).

24. D. Lee et al., “Bone Substitution Material and a Method of Its Manufacture”, U.S. Pat. No. 6,214,368 B1 (Apr. 10, 2001).

25. F. H. Lin et al., “α-TCP/HAP Biphasic Cement and Its Preparing Process”, U.S. Pat. No. 6,338,752 B1 (Jan. 15, 2002).

26. J. Carpena et al., “Method for Making Apatite Ceramics, In Particular for Biological Use”, U.S. Pat. No. 6,338,810 (Jan. 15, 2002).

27. M. Akashi et al., “Hydroxyapatite, Composite, Processes for Producing These, and Use of These”, U.S. Pat. No. 6,395,037 B1 (May 28, 2002).

28. B. Edwards et al., “Porous Calcium Phosphate Cement”, U.S. Pat. No. 6,547,866 B1 (Apr. 15, 2003).

29. P. Higham, “Calcium Phosphate Composition and Method of Preparing Same”, U.S. Pat. No. 6,558,709 B2 (May 6, 2003).

30. A. Gertzman et al., “Malleable Paste for Filling Bone Defects”, U.S. Pat. No. 6,030,635 (Feb. 29, 2000).

31. F. Dorigatti et al., “Biomaterials for Bone Replacements”, U.S. Pat. No. 6,533,820 B2 (Mar. 18, 2003).

32. S. T. Liu et al., “Resorbable Bioactive Phosphate Containing Cements”, U.S. Pat. No. 5,262,166 (Nov. 16, 1993).

33. Y. Hakamatsuka et al., “Method of Preparing Calcium Phosphate”, U.S. Pat. No. 5,322,675 (Jun. 21, 1994).

34. M. Hirano et al., “Calcium Phosphate Granular Cement and Method for Producing Same”, U.S. Pat. No. 5,338,356 (Aug. 16, 1994).

35. A. Imura et al., “Tetracalcium Phosphate-Based Materials and Processes for Their Preparation”, U.S. Pat. No. 5,536,575 (Jul. 16, 1996).

36. M. Fulmer et al., “Reactive Tricalcium Phosphate Compositions”, U.S. Pat. No. 5,709,742 (Jan. 20, 1998).

37. O. Iwamoto, et al., “Hardening Material”, U.S. Pat. No. 5,092,888 (Mar. 3, 1992).

### SUMMARY OF THE INVENTION

The present invention provides novel compositions that can be used for preparation of bio-compatible and bio-resorbable scaffolds. The invention also provides a solid freeform fabrication (SFF) method for making scaffolds from these compositions. As a specific example of a preferred embodiment, the SFF method for preparing a scaffold from a rapid setting calcium phosphate precursor composition comprises the following four steps:

Step (A) involves preparing a mixture of dry solid powder precursors for producing a calcium phosphate mineral composition, with the precursors comprising a calcium source and a phosphoric acid source free of uncombined water. Step (B) involves preparing a fluid component preferably at a pH in the range of 6-11, wherein the liquid component preferably comprises a member selected from the group consisting of phosphate and carbonate and is further preferably from about 15 to 70 weight percent of the total composition. Step (C) includes operating a material deposition system comprising a liquid deposition device for dispensing the fluid component and a separate solid powder-dispensing device for dispensing the solid powder precursors, respectively, to selected locations on a target surface of an object-supporting platform, wherein the dispensed fluid component and dispensed powder react to form the calcium phosphate composition. Step (D) involves, during the operating step (C), moving the deposition system and the object-supporting platform relative to one another in a plane defined by first and second (X- and Y-) directions and along a third (Z-) direction perpendicular to the X-Y plane to form the calcium phosphate composition into a scaffold.

More generally speaking, Steps (A) and (B) involve preparation of two separate parts of precursor reactants (hereinafter referred to as a two-part or two-component formulation): one comprising a dry component of solid powder particles (no liquid involved) and the other comprising a fluid component (possibly containing solutes dissolved in water or ultra-fine particles dispersed in water). Between these two components (dry and wet) there are contained at least one calcium source and one phosphate source, plus other active reactants or non-reactive additives. When combined, the two components react to form an apatite (particularly hydroxyapatite and its derivatives such as carbonated hydroxyapatite). The powder component and the liquid component are separately dispensed, at small quantities at a time, to produce a small mixture mass (a bead or segment with a size preferably smaller than 50 μm, further preferably smaller than 10 μm, and most preferably smaller than 1 μm). These small mixture masses are dispensed and deposited onto selected spots (or “points”) of a target surface on a spot-by-spot or point-by-point basis. The beads or segments at neighboring are combined to form an integral mass with a desired cross-sectional shape for a constituent layer of a multi-layer, 3-D scaffold structure. The steps are then repeated to form successive layers of the scaffold, with a preceding layer being bonded or adhered to a subsequent layer.

Preferably, the moving step includes the steps of (D1) moving the deposition system and the platform relative to one another in a direction parallel to the X-Y plane to form a first layer of the dispensed powder particles and fluid component on the target surface preferably in a point-by-point fashion; (D2) moving the material deposition system and the platform away from one another in the Z-direction by a desired layer thickness; and (D3) after the portion of the first layer adjacent to the deposition system has substantially solidified, dispensing a second layer of the powder and the fluid components onto the first layer to induce a chemical reaction between the dispensed powder component and fluid component while simultaneously moving the platform and the deposition system relative to one another in a direction parallel to the X-Y plane, whereby the second layer solidifies and adheres to the first layer.

The above steps may be repeated to produce a multiple-layer scaffold in a layer by layer fashion. Specifically, the method includes additional steps of forming multiple layers of the powder precursor and the fluid component on top of one another by repeated dispensing and depositing of the powder precursor and the fluid component from the deposition system as the platform and the deposition system are moved relative to one another in a direction parallel to the X-Y plane. The deposition system and the platform are moved away from one another in the Z-direction by a predetermined layer thickness after each preceding layer has been formed. Further, the depositing of each successive layer is controlled to take place after the deposited fluid component and the powder precursors in the preceding layer immediately adjacent the deposition system have substantially reacted and solidified.

In another example, the composition is comprised of dahllite, an analogs thereof, or otherwise carbonate-substituted form of hydroxyapatite (dahllite-like composition). Again, the composition can be prepared in two parts, one in a dry powder state and the other in a wet fluid state. The powder particles should preferably have an average particle size of two (2) μm or smaller, more preferably 0.5 μm or smaller, and most preferably 0.1 μm (or 100 nm) or smaller. The two parts can be separately dispensed and deposited to selected spots on a target surface where they mix and react with each other to be come hardened. These mixing and reacting steps of both dry and wet components at selected spots (“points”) can be conducted in such a fashion that a scaffold is constructed essentially point by point and layer by layer. The compositions, once mixed at a selected spot, harden usually in less than four minutes and preferably in less than two minutes (with sufficiently small powder particles), into polycrystalline structures that, if so desired, can be further cured subsequent to layer manufacturing.

### BRIEF DESCRIPTION OF THE DRAWINGS

FIG. 1 Apparatus that can be used in a powder-liquid co-deposition-based solid freeform fabrication of a 3-D scaffold.

### DESCRIPTION OF PREFERRED EMBODIMENTS

Freeform Fabrication and Material Requirements:

Basically, the present invention entails a unique, novel, and non-obvious combination of a two-part material formulation and a novel solid freeform fabrication process (SFF) to form a highly useful and effective method for constructing a scaffold point by point (spot by spot, bead by bead, or segment by segment) and layer by layer. The process is executed in an automated fashion under the control of a computer-aided design (CAD) computer.

The formulation includes a dry powder part (normally a mixture of ultra-fine powder particles) and a liquid component. The two parts (or reactants) are separately prepared in two separate reservoirs. They will not be mixed with each other until they are needed to form a “point” (spot, bead, or segment) of a layer of a multi-layer scaffold structure. At the needed moment, the two parts are introduced through two separate channels to combine at a selected spot on a target surface, where they react to form a calcium phosphate or hydroxyapatite composition. The process proceeds to co-deposit the two reactants at a second, normally neighboring, spot where they react to form the desired composition. This second spot is normally bonded or adhered to the first spot. These procedures are repeated until the first layer is built. The reaction must proceed sufficiently fast that the materials at the deposition spots will not flow or spread up to any significant extent to compromise the dimensional accuracy of a layer. The reaction must also be fast enough so that the deposited materials in the first layer are essentially solidified or sufficiently rigid and strong to support the mass of a second layer (and successive layers) before a second layer is deposited onto the first layer.

The above considerations imply that prior art slow-setting precursor compositions to calcium phosphate or apatite material may not be suitable for use in a solid freeform fabrication. The freeform fabrication process will not work at all if the deposited materials can not be solidified immediately upon deposition. The fabrication process will go very slow if it takes a very long time for a layer to become sufficiently rigid and strong for supporting its own weight and the weight of successive layers. This is because one would have to wait a long time before beginning to build a subsequent layer and a typical scaffold may be composed of tens or hundreds of thin layers. Thin layers are preferred over thick layers in order to achieve a better dimensional accuracy in a freeform fabrication part. The selection or development of proper reactant materials is essential to the success of a SFF process for constructing a scaffold based on hydroxyapatite (HAP), and its derivatives or variants. After an extensive and in-depth study, we have found several particularly effective two-part material formulations that can be used in a novel two-dispenser material deposition system for co-deposition of a liquid component and a powder component, which are dispensed separately from two separate channels.

Deposition Methods and Devices

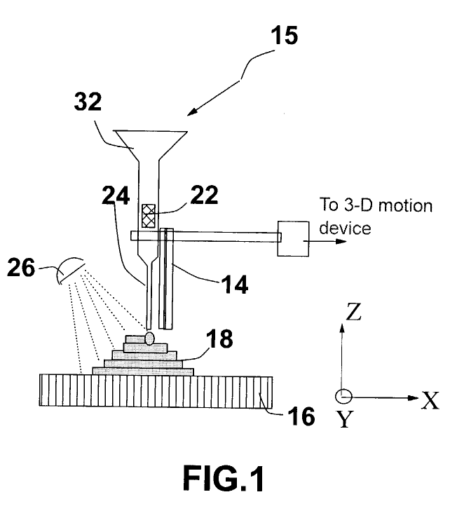

FIG. 1 illustrates one possible apparatus that can be used to practice the present invention for making scaffolds in a fully automated manner. This apparatus is equipped with a computer for creating a drawing or image of a scaffold and, through a hardware controller (including signal generator, amplifier, and other needed functional parts) for controlling the operation of other components of the apparatus. One of these components is a material deposition system which comprises a liquid droplet deposition device 14 and a powder-dispensing device 15. Other components include an object-supporting platform 16, optional temperature-regulating means (e.g., a heat source such as an infrared lamp 26, ultra-violet lamp, forced-convention hot-air blower, etc.) and pump means (not shown) to control the atmosphere of a zone surrounding the platform (if so desired) where a scaffold 18 is being built, and a three dimensional movement system (not shown) to position the platform 16 with respect to the material deposition system in a direction on an X-Y plane and in a Z-direction as defined by the rectangular coordinate system shown in FIG. 1.

There are a broad array of liquid droplet deposition devices that can be incorporated in the material deposition system for practicing the presently invented method. One type of deposition devices is a thermal ink jet print-head. A device of this type operates by using thermal energy selectively produced by resistors located in capillary filled ink channels near channel terminating orifices to vaporize momentarily the ink and form bubbles on demand. Each temporary bubble expels an ink droplet and propels it toward a target surface of the object platform.

Another useful and preferred liquid droplet deposition device is a piezoelectric activated ink jet print-head that uses a pulse generator to provide an electric signal. The signal is applied across piezoelectric crystal plates, one of which contracts and the other of which expands, thereby causing the plate assembly to deflect toward a pressure chamber. This causes a decrease in volume which imparts sufficient kinetic energy to the ink in the print-head nozzle so that one ink droplet is ejected through an orifice.

A liquid droplet deposition device may be a planar high-density array, drop-on-demand ink jet print-head, which typically comprises a print-head body formed with a multiplicity of parallel ink channels. The channels contain liquid compositions and terminate at corresponding ends thereof in a nozzle plate in which are formed orifices. Ink droplets are ejected on demand from the channels and deposited on selected spots of a target surface, which could be a previous layer of a scaffold being built or a surface of the object platform.

Alternatively, the liquid deposition device may be simply a plurality of separate droplet deposition devices, each having only one or two channels. Preferably, at least one of the channels is used to deposit a material such as wax or water-soluble polymer for building the necessary support structure (to support those features of a scaffold structure that can not support themselves during a layer-additive process, such as overhangs and isolated islands).

Preferably, a portion of the liquid deposition device is provided with temperature-controlled means (not shown) to ensure that the material remains in a flowable state while residing in a reservoir, pipe, or channel prior to being dispensed. Heating and cooling means (e.g., heating elements, cooling coils, thermocouple, and temperature controller; not shown) may be provided to a region surrounding the platform 16 to control the solidification behavior of the material on the platform.

In FIG. 1, the powder delivery device 15 comprises a hopper 32 to receive powder particles from a powder supply. More than one hopper may be used to receive two or more types of powders if so desired. Alternatively, a plurality of powder feeders may be combined to provide the capability of supplying and dispensing a mixture of different powders at a desired proportion. The received powder particles flow downward into a capillarity tube 24 which has a discharge orifice at the bottom end. A piezoelectric actuator element 22 is disposed in a proper position on or above the capillarity tube to create an ultrasonic wave or vibration to the tube. It has been found that micron- or nano-scaled powder particles can be discharged from the orifice at a controlled rate when the vibration intensity or frequency exceeds a threshold valve. Such a mechanism serves as an ON-OFF valve that can be controlled in real time by a computer through a proper power supply, signal generator and amplifier circuit.

It may be noted that a SFF process that involves the use of liquid droplets to bind together powder particles was disclosed by one of the applicants (Jang) and co-workers (Jang, Huang, and Zhong, U.S. Pat. No. 6,401,002, Jun. 4, 2002). This process was used primarily for making a multi-color or multi-material object, not a macro-porous scaffold. The liquid droplet dispensing device cited in '002 was used to bind powder particles together and to provide color dyes to powder particles for forming a colorful 3-D object for concept modeling or rapid prototyping applications. The powder particles and the dye-containing liquid did not react to form a reaction product. The method of the subject invention is fundamentally different and distinct from that of '002.

Material Compositions or Formulations:

The powder component of a two-part formulation for forming HAP or its derivative typically comprises a calcium source and a phosphate source. Calcium compounds such as CaCO₃, CaO and Ca(OH)₂ may be used as the calcium source, and phosphorus compounds such as P₂O₅, H₃PO₄, NH₄H₂PO₄ and (NH₄)₂HPO₄ used as the phosphorus source. Alternatively, compounds containing both of Ca and P, such as CaHPO₄.2H₂O, CaHPO₄, Ca(H₂O₄)₂ and Ca₂P₂O₇ may be used as the starting materials.

One particularly useful ingredient of the powder component of a two-part formulation is tetra-calcium phosphate, represented by the formula of Ca₄P₂O₉. The process for preparing tetra-calcium phosphate used in the present invention is not particularly critical and is well known in the art. The procedures for preparing tetra-calcium phosphate may vary according to the desired combination of the starting compounds. For instance, one may use a dry method comprising mixing γ-Ca₂P₂O₇ (obtained by calcining CaHPO₄.2H₂O) with CaCO₃ and calcining the mixture. This reaction is expressed by the following equations:

2CaHPO₄.2H₂O→γ-Ca₂P₂O₇+3H₂O

Ca₂P₂O₇+CaCO₃→Ca₄P₂O₉₊₂CO₂.

If the obtained Ca₄P₂O₉ is calcined at a temperature higher than 1200° C. and rapidly cooled outside the furnace (but preferably in a low-moisture or vacuum environment) or cooled in a nitrogen atmosphere, pure tetra-calcium phosphate can be obtained without an undesired side reaction of direct conversion to hydroxyapatite [Ca₁₀(PO₄)₆(OH)₂].

Another ingredient of the powder component is calcium phosphate having a Ca/P atomic ratio lower than 1.67. This Ca/P atomic ratio is a preferred for the purpose of forming fast-curing hydroxyapatite. Known calcium phosphate can be used without any particular limitation, if the Ca/P atomic ratio is lower than 1.67. F or example, Ca(HPO₄)₂.2H₂O, CaHPO₄.2H₂O, CaHPO₄, Ca₈H₂(PO₄)₆.5H₂O, Ca₃(PO₄)₂ and Ca₂P₂O₇ are preferred materials. Particularly preferred are CaHPO₄.2H₂O and CaHPO₄ because the reaction rate is high and the mechanical properties of the hardened composition are improved.

The powder component, containing the above-mentioned two ingredients, if dispensed and mixed with the deposited water-containing liquid component, form hydroxyapatite. For example, HAP may be formed by the following reaction when CaHPO₄.2H₂O is used as the calcium phosphate: 2 Ca₄P₂O₉+2 CaHPO₄.2H₂O+water→Ca₁₀(PO₄)₆(OH)₂₊₂H₂O.

The particle sizes of tetra-calcium phosphate and calcium phosphate in the powder mixture are important parameters in dictating the setting time of the two-part formulation when the powder and the liquid components are dispensed to the same spots during the freeform fabrication process. When the average particle size of the tetra-calcium phosphate and the calcium phosphate having a Ca/P atomic ratio lower than 1.67 were reduced from 13 μm to 4 μm, the setting time was shortened from approximately 45 minutes to 4 minutes. The setting time was further reduced to below 2 minutes with an average particle size smaller than 1 μm.

In one preferred embodiment, the liquid component of the two-part formulation is a colloidal aqueous solution comprising solid colloid particles dispersed in an aqueous medium. Various colloidal aqueous solutions of this type are known. In general, these aqueous solutions are divided into “sols” comprising inorganic solid particles dispersed in an aqueous medium and “latexes” comprising organic polymer particles dispersed in an aqueous medium. Any of known sols and latexes can be used in practicing the present invention without any particular limitation. A sol comprising inorganic oxide particles, such as a silica or alumina, or a so-called polymer latex such as a latex of polymethyl methacrylate or polystyrene, may be used as the liquid component in the present invention. A sol comprising inorganic oxide particles may be used if the crystallinity, the bio-compatibility, and the increase in compressive strength of the resulting hydroxyapatite are important considerations. If the safety to a living body and the storage stability are important, a silica sol or alumina sol is especially preferred.

The concentration of the solid colloid particles in the aqueous solution used in the present invention may vary with different kinds of colloid particles. However, in general, the preferred concentration is from 5 to 60% by weight, especially from 10 to 50% by weight. The mixing ratio between the powder component and the liquid component may be selected so that a viscosity suitable for freeform fabrication is attained without compromising the strength of the resulting HAP. The powder/liquid mixing weight ratio is preferably in the range of from 0.5 to 5, especially from 2 to 4. Hydroxyapatite, silica, calcium fluoride, titanium dioxide, calcium hydroxide, alumina, sodium phosphate or ammonium phosphate can be added to the formulation so as to adjust the setting time and the strength of the material.

Similar examples of two-component HAP precursor formulations include those disclosed by Brown and Chow (e.g., U.S. Pat. Nos. Re. 33,221 and Re. 33,161). The preferred major components of the calcium phosphate cement of Brown and Chow are tetra-calcium phosphate (TTCP) and dicalcium phosphate anhydrous (DCPA) or dicalcium phosphate dihydrate (DCPD). These ingredients react in an aqueous environment to form hydroxyapatite. But, preferably, the calcium (Ca) to phosphate (PO₄) molar ratio in the prepared tetra-calcium phosphate is below 2. Further, the tetra-calcium phosphate is preferably kept under a substantially anhydrous environment during its synthesis, quenching, particle size reduction processes, and storage. If the prepared tetra-calcium phosphate has a molar Ca/P ratio above 2, calcium oxide is believed to be present in the material as an impurity phase. When such a tetra-calcium phosphate sample is used in the cement, the rapid dissolution of the CaO presumably causes the pH of the formulation (with the powder and liquid parts combined) to rise substantially above pH 8.5 (but below 12), which impedes the setting reaction.

For use in the present invention, the two-part calcium phosphate composition proposed by Brown and Chow may comprise a solid powder part and a liquid part. The two parts, when combined, self-harden substantially to hydroxyapatite at ambient temperature. The powder part comprises tetra-calcium phosphate and at least one other sparingly soluble calcium phosphate compound, wherein the tetra-calcium phosphate is prepared from a starting mixture of one or more sources of calcium, phosphorous and oxygen which mixture has a calcium to phosphorous ratio of less than 2. Again, particle sizes of the powder mixture are important in dictating the setting time and strength of the combined two-part composition. An average particle size of smaller than 4 μm is preferred, that smaller than 1 μm is further preferred, and that smaller than 100 nm is most desirable. Alternatively, a sparingly soluble calcium phosphate compound may be dissolved or dispersed in an aqueous medium as a part of the liquid part, provided the viscosity of the resulting liquid part is not excessively high to avoid compromising the liquid droplet ejection operation using an inkjet printhead.

The HAP derivative formulations that can be used in the present freeform fabrication of scaffolds include compositions that are comprised of dahllite, analogs thereof, or otherwise carbonate-substituted forms of hydroxyapatite (dahllite-like compositions). The compositions can be prepared in two parts, one in a dry powder state and the other in a wet fluid state. The powder particles should preferably have an average particle size of two (2) μm or smaller, more preferably 0.5 μm or smaller, and most preferably 0.1 μm (or 100 nm) or smaller. The compositions (with the dry and liquid parts dispensed to the same spots) harden, normally in less than four minutes. With the average particle sizes smaller than 0.5 μm, the compositions could harden in less than two minutes into polycrystalline structures.

One specific example is a two-part calcium phosphate cement formulation that, when mixed, is capable of hardening and forming an integral mass, which is approximately 2 to 10 wt % carbonate-substituted hydroxyapatite that has a calcium/phosphate molar ratio of about 1.33 to 2.0. The two-part calcium phosphate cement formulation contains a dry powder part and a wet fluid part. The powder part comprises ultra-fine dry powder particles, with an average particle size smaller than 4 μm in diameter. The powders include primarily a partially neutralized phosphoric acid, a calcium phosphate source, and calcium carbonate in an amount ranging front about 9.33 to 70 wt % of the dry powder part. The wet fluid part contains a physiologically acceptable aqueous lubricant solution, which is either a 0.01 to 2M sodium phosphate solution at pH 6 to 11 or a 0.01 to 2M sodium carbonate solution at pH 6 to 11. The aqueous lubricant solution is present in an amount ranging from about 15 to 50 wt % of the two-part calcium phosphate cement formulation.

The preferred powder particle sizes are 2 μm or smaller. The further preferred average particle sizes are 0.5 μm or smaller and most preferred average particle sizes are 0.1 μm (100 nm) or smaller. With average particle sizes being smaller than 2 μm, the setting time of the dry and wet parts when mixed together is typically four (4) minutes or shorter at a setting temperature of 37° C. in air. The setting time is reduced to approximately two (2) minutes or shorter when the mixture is made from finer particles with an average particle size smaller than 0.5 μm. Still finer particles (100 nm or smaller) only lead to a slightly shorter setting time (less than 2 minutes), but result in improved mechanical properties of the carbonated HAP.

The composition of the carbonated hydroxyapatite may vary. For instance, the calcium/phosphate ratio may vary from 1.33 to 2.0 with 1.67 being the natural ratio. With the ratio smaller than 1.67, there will be a defective lattice structure from the calcium vacancies. For a ratio of 1.33, there will be two calcium ions absent. The extra hydrogens may be up to about 2 hydrogen ions per phosphate, usually not more than about one hydrogen ion per phosphate. The ions will be uniformly distributed throughout the product.

The dry powder reactant typically consists of a phosphoric acid source substantially free of unbound water, an alkali earth metal source (particularly calcium source), optionally crystalline nuclei (particularly hydroxyapatite or calcium phosphate crystals), and calcium carbonate. The wet fluid part or reactant typically comprises a physiologically acceptable lubricant (e.g., water), which may contain various solutes. The dry ingredients may be prepared as a mixture of ultra-fine powders and subsequently combined with the liquid ingredients during freeform fabrication.

Specifically, the phosphoric acid source may be any partially neutralized phosphoric acid, particularly up to complete neutralization of the first proton as in calcium phosphate monobasic. It can consist of orthophosphoric acid, possibly in a crystalline form, which is substantially free of combined water. The acid source will generally be about 15 to 35 weight percent of the dry components of the mixture, more usually 15 to 25 weight percent.

The calcium source could play a dual role of providing calcium and acting as a neutralizing agent. The desired final product depends on the relative ratios of calcium and phosphate. Calcium sources generally include counter-ions such as carbonate and phosphate. Dual sources of calcium and phosphate such as tetra-calcium phosphate or tri-calcium phosphate are particularly useful. The proportion of tetra-calcium phosphate or tri-calcium phosphate in the mixture may typically lie from about 0 to 70 weight percent, more preferably from about 0 to 40 weight percent, and most preferably from about 2 to 18 weight percent of dry weight of the dry components of the mixture.

One major advantage of having calcium carbonate being present to serve as a source of calcium and carbonate is that it also serves to neutralize the acid and, hence, the reaction results in relatively little temperature rise. However, there is substantial evolution of gas which must be released during mixing if micro pores (<50 μm) are not desired. Calcium carbonate will be present in the mixture from about 2 to 70 weight percent, preferably from about 2 to 40 weight percent, and most preferably from about 2 to 18 weight percent of dry weight of the dry components of the mixture. Calcium hydroxide may also be present in the mixture from about 0 to 40 wt. %., preferably from about 2 to 25 wt. %, and most preferably from about 2 to 20 wt. %.

Preferably all the dry powder ingredients are combined to form the dry powder part of the two-part composition. Alternatively, one may choose to dissolve a small amount of a dry powder ingredient in the liquid (wet lubricant) part to adjust the consistency of the wet fluid part of the two-part composition. This could also help to improve the uniformity of the various components, dry and wet, when combined together to form a reactive mass. Various solutes may be included in the wet fluid part. For instance, a gel or colloid, which has as a solute alkali metal hydroxide, acetate, phosphate, or carbonate, particularly sodium, more particularly phosphate or carbonate, may be added at a concentration in the range of 0.01 to 2 M, particularly 0.05 to 0.5 M, and at a pH in the range of about 6-11, more usually about 7-9, particularly 7-7.5.

Various dry powders may be size-reduced to 2 μm (or preferably 0.5 μm and further preferably 100 nm) or smaller via ball milling. The high-energy planetary ball mill available from Nanotek Instruments, Inc. (Fargo, N. Dak.) is capable of reducing various ceramic powders down to nanometer scales. The dry components may be ball-milled separately and then combined to form a mixture or, alternatively, are combined to form a mixture of dry powders, which are then ball-milled to the desired size scales. The particle sizes, to a great extent, dictate the setting time of the resulting mixture of dry and wet components.

By varying the proportion of liquid lubricant, particularly water, added to the subject mixtures, the fluidity of the composition can be adjusted. Other water soluble and compatible liquids that are pharmacologically acceptable may be added to the wet fluid part of the two-part composition. These may include alkanols, more particularly polyols, such as ethylene glycol, propylene glycol or glycerol. These diluents or thickening agents may be present in less than about 10 volume percent in an appropriate medium. The liquid will generally be from about 15 to 50, more usually from about 20 to 35 weight percent of the entire composition, dry and wet components together during freeform fabrication.

After being dispensed to mix at desired spots, the compositions will undergo chemical reactions to become hardened. During hardening, crystal growth occurs and the product becomes an integral mass. The resulting mass will have a composition that contains structurally incorporated carbonate in the apatite structure. The carbonate proportion lies between about 2% and about 10% carbonate by weight, usually between 2.5% to 7%, and optimally between about 4% to about 6% carbonate by weight.

The un-cured compositions could have a pH in the range of about 5.5-8.5, but usually in the range of about 6-7.5. They can be cured in an environment having a temperature in the range of about 0-45° C., usually 20-40° C. A heat source may be present to accelerate or facilitate the hardening process of the combined formulation after dispensing. The compositions are bio-compatible, having low or no toxicity when prepared in accordance with the above-described methods. They are readily resorbable in vivo and, hence, the set mass could be gradually replaced by natural bone.

For some clinical applications, it may be advantageous to include additional components into the mixture during the formation of the carbonated hydroxyapatite. Examples of useful components are pharmacologically active agents, proteins, polysaccharides, and other biocompatible polymers. Of particular utilization value are proteins involved in skeletal structure such as various forms of collagen (fibrin, fibrinogen, keratin, tubulin, elastin, etc.) or structural polysaccharides, such as chitin. Pharmacologically active agents that might be added include drugs that enhance bone growth, serve as a variety of cell growth factors, or act as anti-inflammatory or anti-microbial agents. Examples of such agents include bone morphogenetic protein (BMP), cartilage induction factor, platelet derived growth factor, and skeletal growth factor.

Pharmacologically active agents or structural proteins may be added as an aqueous dispersion or solution. The protein usually will be present in from about 1-10 wt % of the aqueous dispersion. After hardening, the resulting composition will contain the protein in from about 0.01 to 10, usually from about 0.05 to 5 weight percent. By varying the proportions of the reactants, one can obtain compositions with varying and predictable rates of resorption in vivo. In sum, a clinician can add drug and inorganic components to the invented compositions in order to practice an implantable time-release delivery platform for drugs, inorganic mineral supplements, or the like.

One specifically preferred embodiment of the present invention is the preparation of carbonated hydroxyapatite by a process whereby a calcium source (at least one component of which is calcium carbonate) and an acidic phosphate source (optionally comprised of orthophosphoric acid crystals substantially free of uncombined water) are mechanically mixed for a sufficient length of time to allow for a partial reaction between the calcium source and acidic phosphate source to occur. The partially reacted composition is in the form of a fine powder with average particle size smaller than 2 μm (preferably smaller than 0.5 μm and most preferably smaller than 100 nm). During the solid freeform fabrication process, the powder can be dispensed and mixed with a dispensed physiologically suitable lubricant fluid component to achieve a substantially complete reaction between the reactants.

The calcium source used in the above process will typically include a mixture of tetra-calcium phosphate (TCP) and calcium carbonate with the former typically present in from about 55 to 75 wt. %, or more usually 60-70 wt. %, and the latter typically present in from about 1 to 40 wt. %, or more typically 2 to 18 wt. % of the dry weight of the total reaction mixture. The acid phosphate source will be about 15 to 35, or more preferably 15 to 25 wt. % of the dry weight of the reaction mixture.

Alternatively, the composition may typically include a mixture of tri-calcium phosphate (TrCP), calcium carbonate (CC), and calcium hydroxide (CH) with TrCP typically present in from about 50 to 90 wt. %, or more usually 75 to 90 wt. %, CC typically present from about 1 to 40 wt. % or more usually 2 to 18 wt. %, and CH typically present from about 0 to 40 wt. % or more usually 2 to 20 wt. % of the dry weight of the total reaction mixture. The acid phosphate source for this mixture will be about 5 to 35 wt. % or more usually 5 to 25 wt. % of the dry weight of the reaction mixture. A fluoride source may be added to the mixture in an amount from about 0 to 4 wt. %, preferably 3 to 4 wt. % of dry weight.

After the dry ingredients are combined, the reactants will be placed in intimate contact by ball milling for the purposes of reducing the particle sizes and facilitating partial reactions between selected ingredients, if so desired. The product that has undergone a partial reaction will require less dispensed liquid lubricant and will result in a reduced setting time of the final mixture.

In sum, a two-component formulation can be devised in such a way that certain ingredients or reactants are in the form of a fine powder and the remaining ingredients in the form of a fluid (liquid, melt, solution, sol, etc.). The fine powder component and the liquid component are stored in separate containers and, during the powder-liquid co-deposition-based solid freeform fabrication process, are separately dispensed to essentially the same spots on a target surface. The resulting mixture, in minute “beads”or “segments”, forms a layer of a scaffold in an essentially “point-by-point” or “spot-by-spot” basis. Once a layer is built and the materials deposited have reacted to the extent that they are of sufficient rigidity and strength to support their own weight (without excessive flow or deformation) and the weights of next layers, a second layer is dispensed and deposited. These steps are repeated until a multi-layer macro-porous scaffold containing desired pore sizes and porosity level is fabricated, all under the control of a computer.

The material dispensing steps are carried out in a predetermined pattern that is governed by the shape of a 3-D scaffold to be formed. The dispensed materials (powder plus fluid) are deposited in multiple layers which solidify and adhere to one another to build up a 3-D shape. The predetermined pattern is such that the resulting 3-D scaffold shape contains macro pores (with a size greater than 50 μm, preferably greater than 100 μm, and most preferably greater than 200 μm), which are preferably interconnected to allow for easy access of the in-growing cells when used for tissue repair or re-generation.

Preferably, these procedures are accompanied by a micro-pore forming procedure, simultaneously with and/or after the depositing procedures, to form micro-pores (<50 μm) in addition to the macro-pores (>50 μm). The macro-pores provide sufficient space for extracellular matrix regeneration and minimal diffusional constraints of cells, growth factors, nutrients, and metabolic waste. The micro-pores provide (1) increased surface areas for cells to cling to, (2) additional parameter to control the physical density of the scaffold, (3) additional space for cells, growth factors, nutrients and metabolic waste to migrate through, and (4) enhanced bio-degradation and bio-resorption rates, if so desired.

The formation of micro pores in a HAP matrix can be accomplished in several ways. For instance, micro pores may be generated by adding a pore-foaming agent (e.g., NaCl salt or sugar) to the powder component of the two-part formulation. After a complete macro-porous 3-D scaffold is made by using the presently invented procedures, the scaffold is immersed in a water bath. This allows the salt or sugar component to leach out, leaving behind micro pores in the scaffold. Such a scaffold is both micro-porous and macro porous. In general, suitable leachable solids include but are not limited nontoxic leachable materials selected from the group consisting of salts (i.e. sodium chloride, potassium chloride, calcium chloride, sodium tartrate, sodium citrate, and the like) biocompatible mono- and di-saccharides (i.e. glucose, fructose, dextrose, maltose, lactose and sucrose), polysaccharides (i.e. starch, alginate), water soluble proteins (i.e. gelatin and agarose). Generally all of these materials will be chosen to have an average diameter 6 of less than about 50 μm. The particles will generally constitute from about 1 to about 50 volume percent of the total volume of the final HPA composition. The leachable materials can be removed by immersing the scaffold with the leachable material in a solvent in which the particle is soluble for a sufficient amount of time to allow leaching of substantially all of the particles, but which does not dissolve or detrimentally alter the scaffold. The preferred extraction solvent is water, most preferably distilled-deionized water.

In addition, any common foaming process may be adapted for creating micro pores. Examples include the use of a physical or chemical blowing agent in the matrix. The blowing agent may be allowed to become activated during or after the deposition procedures. The carbon dioxide gas that is naturally produced in the formation process of some carbonated HPA mentioned earlier (e.g., with precursor ingredients containing calcium carbonate) can be advantageously used to generate micro pores.

What is claimed is:

1. A method for preparing a scaffold from a rapid setting calcium phosphate composition, said method comprising:
(A) preparing a dry solid powder mixture of precursors for producing a calcium phosphate mineral composition, said precursors comprising a calcium source and a phosphoric acid source free of uncombined water;
(B) preparing a fluid component at a pH in the range of 6-11, wherein said fluid component comprises a member selected from the group consisting of phosphate and carbonate and is from about 15 to 70 weight percent of the total composition;
(C) operating a material deposition system comprising a liquid deposition device for dispensing said fluid component and a solid powder-dispensing device for dispensing said solid powder precursors to selected locations on a target surface of an object-supporting platform, wherein said dispensed fluid component and dispensed powder precursors react to form said calcium phosphate composition; and
(D) during said operating step (C), moving said deposition system and said object-supporting platform relative to one another in a plane defined by first and second directions and along a third direction perpendicular to said plane to form said calcium phosphate composition into said scaffold.

2. The method according to claim 1, wherein said member selected from the group consisting of phosphate and carbonate is present in said liquid component in a concentration ranging from 0.05 to 0.5M and said pH of said fluid component is in the range of about 7 to 9.

3. The method according to claim 1, wherein said calcium source comprises at least one of a member selected from the group consisting of tetra-calcium phosphate and calcium carbonate.

4. A method for preparing a scaffold from a rapid setting calcium phosphate composition, said method comprising:
(A) preparing a dry solid powder mixture of precursors for producing a calcium phosphate mineral composition, said precursors comprising a calcium source comprising tetra-calcium phosphate and calcium carbonate and a phosphate source comprising at least one of mono-calcium phosphate and orthophosphoric acid free of uncombined water;
(B) preparing a fluid component comprising a member selected from the group consisting of sodium phosphate and carbonate in a concentration ranging from 0.05 to 0.5M, said fluid component at a pH in the range of 6-11, wherein said fluid component is from about 15 to 70 weight percent of the total composition;
(C) operating a material deposition system comprising a liquid deposition device for dispensing said fluid component and a solid powder-dispensing device for dispensing said solid powder precursors to selected locations on a target surface of an object-supporting platform, wherein said dispensed fluid component and dispensed powder precursors react to form said calcium phosphate composition; and
(D) during said operating step (C), moving said deposition system and said object-supporting platform relative to one another in a plane defined by first and second directions and along a third direction perpendicular to said plane to form said calcium phosphate composition into said scaffold.

5. The method according to claim 4, wherein said fluid component comprises sodium phosphate at a pH in the range of about 7 to 9.

6. A solid freeform fabrication method for producing a scaffold from a two-part calcium phosphate cement formulation that, when mixed, is capable of hardening and forming an integral mass, wherein said integral mass is approximately 2 to 10 wt % carbonate-substituted hydroxyapatite that has a calcium/phosphate molar ratio of about 1.33 to 2.0, said method comprising:
(A) preparing, as the first part of said cement formulation, a mixture of ultra-fine dry powder ingredients, comprising a partially neutralized phosphoric acid, a calcium phosphate source, and calcium carbonate in an amount ranging from about 9.33 to 70 wt % of said mixture of dry powder ingredients;
(B) preparing, as the second part, a physiologically acceptable aqueous fluid solution component selected from the group consisting of 0.01 to 2M sodium phosphate solution at pH 6 to 11 and 0.01 to 2M sodium carbonate solution at pH 6 to 11, wherein said aqueous fluid solution is present in an amount ranging from about 15 to 50 wt % of the two-part calcium phosphate cement formulation;
(C) operating a material deposition system comprising a liquid deposition device for dispensing said fluid solution and a solid powder-dispensing device for dispensing said solid powder ingredients to selected locations on a target surface of an object-supporting platform, wherein said dispensed fluid component and dispensed powder ingredients react to form said carbonate-substituted hydroxyapatite; and
(D) during said operating step (C), moving said deposition system and said object-supporting platform relative to one another in a plane defined by first and second directions and along a third direction perpendicular to said plane to form said dispensed two-part formulation into said scaffold.

7. The method according to claim 6, wherein said partially neutralized phosphoric acid source is Ca(H₂PO₄)₂H₂O.

8. The method according to claim 6, wherein said calcium phosphate source is tri-calcium phosphate.

9. A solid freeform fabrication method for producing a scaffold from a two-part calcium phosphate cement formulation that, when mixed, is capable of hardening and forming an integral mass in less than 4 minutes, wherein said integral mass is approximately 2 to 10 wt % carbonate-substituted hydroxyapatite that has a calcium/phosphate molar ratio of about 1.33 to 2.0 and is bio-compatible, said method comprising:
(A) preparing, as the first part of said two-part formulation, a mixture of ultra-fine dry powder ingredients, comprising a partially neutralized phosphoric acid, a tri-calcium phosphate, and calcium carbonate in an amount ranging from about 9.33 to 40 wt % of said mixture of dry powder ingredients;
(B) preparing, as the second part of said two-part formulation, a physiologically acceptable aqueous fluid component selected from the group consisting of 0.01 to 2M sodium phosphate solution at pH 6 to 11 and 0.01 to 2M sodium carbonate solution at pH 6 to 11, wherein said aqueous fluid component is present in an amount ranging from about 15 to 50 wt % of the two-part calcium phosphate cement formulation;
(C) operating a material deposition system comprising a liquid deposition device for dispensing said fluid component solution and a solid powder-dispensing device for dispensing said solid powder ingredients to selected locations on a target surface of an object-supporting platform, wherein said dispensed fluid component and dispensed powder ingredients react to form said carbon-substituted hydroxyapatite; and
(D) during said operating step (C), moving said deposition system and said object-supporting platform relative to one another in a plane defined by first and second directions and along a third direction perpendicular to said plane to form said dispensed two-part formulation into said scaffold.

10. A method for preparing a calcium phosphate-based macro-porous scaffold, said method comprising:
(A) preparing a mixture of dry solid powder particles in a powder container;
(B) preparing a fluid component in a reservoir separate from said powder container; wherein said powder mixture and said fluid component, separately or in combination, comprise at least a calcium source and a phosphoric acid source;
(C) operating a material deposition system comprising a liquid deposition device for dispensing said fluid component from said reservoir and a solid powder-dispensing device for dispensing said solid powder mixture from said container to selected locations on a target surface of an object-supporting platform, wherein said dispensed fluid and dispensed powder components react to form said calcium phosphate composition; and
(D) during said operating step (C), moving said deposition system and said object-supporting platform relative to one another in a plane defined by first and second directions and along a third direction perpendicular to said plane to form said calcium phosphate composition into said scaffold containing macro pores, greater than 50 μm in size.

11. The method according to claim 1, 4, 6, 9, or 10, wherein the average particle size of said powder is 4 μm or smaller.

12. The method according to claim 1, 4, 6, 9, or 10, wherein the average particle size of said powder is 2 μm or smaller.

13. The method according to claim 1, 4, 6, 9, or 10, wherein the average particle size of said 6 powder is 100 nanometers or smaller.

14. The method according to claim 1, 4, 6, 9, or 10, wherein at least one of said powder component and fluid component comprises a protein in an amount equal to from about 0.1 to 5% by weight as compared with the total weight of calcium phosphate composition.

15. The method as set forth in claim 1, 4, 6, 9, or 10, wherein the moving step includes the steps of:
moving said deposition system and said platform relative to one another in a direction parallel to said plane to form a first layer of said dispensed powder and said dispensed fluid component on said target surface;
moving said material deposition system and said platform away from one another in said third direction by a desired layer thickness; and
after the portion of said first layer adjacent to said deposition system has substantially solidified, dispensing a second layer of said powder and said fluid component onto said first layer to induce a chemical reaction between said dispensed powder and fluid component while simultaneously moving said platform and said deposition system relative to one another in a direction parallel to said plane, whereby said second layer solidifies and adheres to said first layer.

16. The method as set forth in claim 15, comprising additional steps of forming multiple layers of said powder and said fluid component on top of one another by repeated dispensing and depositing of said powder and said fluid component from said deposition system as said platform and said deposition system are moved relative to one another in a direction parallel to said plane, with said deposition system and said platform being moved away from one another in said third direction by a predetermined layer thickness after each preceding layer has been formed and with the depositing of each successive layer being controlled to take place after said deposited fluid component and said powder in the preceding layer immediately adjacent said deposition system have substantially reacted and solidified.

17. The method as set forth in claim 1, 4, 6, 9, or 10, further comprising additional step of exposing said dispensed powder and dispensed fluid component to an energy source of sufficient energy or intensity to facilitate a chemical reaction between said dispensed fluid component and said dispensed powder.

18. The method as set forth in claim 1, 4, 6, 9, or 10, further comprising the steps of:
creating an image of said scaffold on a computer with said image including a plurality of segments or data points defining the scaffold;
generating programmed signals corresponding to each of said segments or data points in a predetermined sequence; and
moving said deposition system and said platform relative to each other in response to said programmed signals.

19. The method as set forth in claim 18, further comprising:
using dimension sensor means to periodically measure dimensions of the scaffold being built;
using a computer to determine the thickness and outline of individual layers of said fluid component and powder precursor deposited in accordance with a computer aided design representation of said scaffold; said computer being operated to calculate a first set of logical layers with specific thickness and outline for each layer and then periodically re-calculate another set of logical layers after comparing the dimension data acquired by said sensor means with said computer aided design representation in an adaptive manner.

20. The method as set forth in claim 1, 4, 6, 9, or 10, further comprising the steps of:
creating an image of said scaffold on a computer, said image including a plurality of segments or data points defining said scaffold;
evaluating the data files representing said scaffold to locate any un-supported feature of the scaffold, followed by defining a support structure for the un-supported feature and creating a plurality of segments or data points defining said support structure;
generating program signals corresponding to each of said segments or data points for both said scaffold and said support structure in a predetermined sequence;
during said deposition step, in response to said programmed signals, moving said deposition system and said platform relative to one another in said plane and in said third direction in a predetermined sequence of movements such that said powder and fluid component are deposited in free space as a plurality of segments or beads sequentially formed so that the last deposited segment or bead overlies at least a portion of the preceding segment or bead in contact therewith to thereby form said support structure and said scaffold.

21. The method as set forth in claim 1, 4, 6, 9, or 10, further comprising steps of operating a material deposition device and moving said platform relative to said deposition device to build a support structure for said scaffold.

22. A method for making bone repair, said method comprising introducing, at a bone site for repair, a scaffold prepared according to the method of claim 1, 4, 6, 9, or 10.

23. The method as set forth in claim 1, 4, 6, 9, or 10, wherein said scaffold contain macro pores that are equal or greater than 100 μm in size.

24. The method as set forth in claim 23, wherein said scaffold further contains micro pores that are equal or smaller than 50 μm in size.

25. The method as set forth in claim 1, 4, 6, 9, or 10, wherein at least one of said powder component or fluid component further contains one agent selected from the group consisting of pharmacologically active agents, proteins, polysaccharides, biocompatible polymers, fibrin, fibrinogen, keratin, tubulin, elastin, chitin, bone growth-enhancing drug, cell growth factors, anti-inflammatory agents, anti-microbial agents, morphogenetic protein (BMP), cartilage induction factor, platelet derived growth factor, skeletal growth factor, and combinations thereof.
